(19) United States
(12) Patent Application Publication
(10) Pub. No.: US 2024/0274227 A1
(43) Pub. Date: Aug. 15, 2024

(54) DNAZYME DESIGN

(71) Applicant: UNIVERSITY OF ESSEX ENTERPRISES LIMITED, Colchester, Essex (GB)

(72) Inventors: Antonio MARCO CASTILLO, Colchester, Essex (GB); Greg BROOKE, Colchester, Essex (GB); Angela PINE, Colchester, Essex (GB)

(21) Appl. No.: 18/561,661

(22) PCT Filed: May 13, 2022
(86) PCT No.: PCT/GB2022/051217

§ 371 Date: Nov. 16, 2023

(30) Foreign Application Priority Data
May 17, 2021 (GB) 2107029.7

(51) Int. Cl. G16B 15/10; G16B 40/00

(52) U.S. Cl. CPC G16B 15/10; G16B 40/00

(57) ABSTRACT

A method for providing at least one DNAzyme for performing a predetermined function on a target string and a method for training computer implemented instructions executable on a processor for providing at least one DNAzyme for performing a predetermined function on a target string are disclosed. The method for providing at least one DNAzyme for performing a predetermined function on a target string includes identifying at least one potential target site of a target string; proposing a plurality of possible DNAzyme sequences which may perform a predetermined function on at least one target site, determining at least one DNAzyme characteristic of each DNAzyme sequence of the plurality of possible DNAzyme sequences utilising a model to indicate a relationship between the at least one DNAzyme characteristic and a predetermined function probability of each DNAzyme sequence of the plurality of possible DNAzyme sequences, and determining if the predetermined function probability each DNAzyme sequence of the plurality of possible DNAzyme sequences are above a predetermined function probability threshold.

Enzymatic nucleic acid molecules (also referred to as deoxyribozymes, DNA enzymes or DNAzymes) are synthetic catalysts constructed from single stranded deoxyribonucleic acid polymers, which can be categorised into either 10-23 or 8-17 enzymatic nucleic acids. Enzymatic nucleic acids consist of a catalytic core flanked by two specificity sequences and have the ability to fold into intricate secondary structures. The catalytic core typically requires a divalent cation, such as Mg2+, Ca2+, Pb2+, as a cofactor to be catalytically active. Currently DNAzymes have a broad range of catalytic functions with one of the most common being ribonuclease activity. The ribonuclease activity of an enzymatic nucleic acid is conducted by a transesterification reaction that occurs through an acid-base catalytic mechanism. The catalytic activity of enzymatic nucleic acid molecules may require the presence of a cofactor, namely a divalent cation which acts as a base in a transesterification reaction. The divalent cation mediates nucleophilic attack of the 2′-hydroxyl group at the adjacent phosphodiester linkage of the target RNA. The target RNA is cleft, due to interaction with a DNAzyme, between an unpaired purine nucleotide (A, G) and a paired pyrimidine nucleotide (U, C) and results in generation of two RNA fragments.

An enzymatic nucleic acid molecule with ribonuclease activity recognises its target via Watson and Crick base pairing of the flanking sequences to the target RNA. Base complementarity recognition of the target by the flanking sequences allows for the selection and design of enzymatic nucleic acids to specific targets and therefore conducts catalysis on a specific RNA. The specificity of enzymatic nucleic acid molecules can be employed to regulate the expression of specific genes via degradation of their transcripts and therefore impart control over certain cellular pathways, particularly those disrupted in a disease state.

Enzymatic nucleic acid molecules have an advantage over other gene expression therapeutics as they function independently of the cellular machinery, are specific to a target RNA and possess a higher degree of chemical and also enzymatic stability. The low cost and ease of synthesis of DNA polymers is also an advantage over other therapeutic methods.

A number of enzymatic nucleic acid molecules have been developed to combat several diseases. Several of these have undergone or are currently undergoing clinical trials for the treatment of nodular basal-cell carcinoma, nasopharyngeal carcinoma, severe allergic bronchial asthma, atopic dermatitis and ulcerative colitis. Furthermore, DNAzymes have been developed as diagnostic tools and biosensors for metal ions and bacteria and have even found use in logic gates, computer circuits and biofuel applications. Given the developmental success of DNAzyme molecules in combatting disease, and their far reaching potential application elsewhere, an effective and efficient method of designing, determining and/or identifying DNAzyme molecules is desirable.

Current approaches in designing DNAzymes for a particular function involve utilisation of a combination of tools which are not specifically designed for use for DNAzymes. Such tools include the Vienna RNA package, for example. Furthermore, approaches to the design of DNAzymes have not been standardised and therefore there exists significant variation in such approaches. Furthermore, for ‘10-23’ DNAzymes the substrate binding arm/flanking regions are matched to regions of a target string or a gene of interest randomly. As any one target/gene can have tens to hundreds of possible cleavage sites for a DNAzyme depending on their size and sequence, designing DNAzyme sequences is often achieved through trial and error. The efficiency of DNAzyme sequences are typically assessed via in vitro cleavage reactions. This approach can be both time consuming and costly. In order to simplify this process, it has been proposed to make use of short fragments of the target RNA however when adopting such an approach the biochemical and structural parameters which would exist physiologically, that affect DNAzyme efficiency, are lost.

It is an aim of the present invention to at least partly mitigate one or more of the above-mentioned problems.

It is an aim of certain embodiments of the present invention to provide an efficient and reliable method for providing at least one DNAzyme for performing a predetermined function on a target string.

It is an aim of certain embodiments of the present invention to reduce the difficulties, costs and/or timescales associated with providing at least one DNAzyme for performing a predetermined function on a target string.

It is an aim of certain embodiments of the present invention to provide a machine learning approach to providing at least one DNAzyme for performing a predetermined function on a target string.

According to a first aspect of the present invention there is provided a method for providing at least one DNAzyme for performing a predetermined function on a target string, comprising the steps of: identifying at least one potential target site of a target string; proposing a plurality of possible DNAzyme sequences which may perform a predetermined function on at least one target site; determining at least one DNAzyme characteristic of each DNAzyme sequence of the plurality of possible DNAzyme sequences; utilising a model to indicate a relationship between the at least one DNAzyme characteristic and a predetermined function probability of each DNAzyme sequence of the plurality of possible DNAzyme sequences; and determining if the predetermined function probability each DNAzyme sequence of the plurality of possible DNAzyme sequences are above a predetermined function probability threshold.

Aptly the method further comprises assorting the plurality of possible DNAzyme sequences into a plurality of groups whereby a first group includes DNAzyme sequences of the plurality of DNAzyme sequences for which the predetermined function probability is known, and a further group includes DNAzyme sequences of the plurality of DNAzyme sequences for which the predetermined function probability is unknown.

Aptly determining if the predetermined function probability of each DNAzyme sequence of the plurality of possible DNAzyme sequences of the first group are above a predetermined function probability threshold includes fitting the model to the at least one DNAzyme characteristic of each DNAzyme sequence of the plurality of possible DNA sequences of the first group and the predetermined function probability of the plurality of possible DNAzyme sequences of the first group.

Aptly determining if the predetermined function probability each DNAzyme sequence of the plurality of possible DNAzyme sequences of the further group are above a predetermined function probability threshold includes providing the at least one DNAzyme characteristic of each DNAzyme sequence of the plurality of possible DNAzyme sequences of the first group to the model to determine the predetermined function probability of each DNAzyme sequence of the plurality of possible DNAzyme sequences of the further group.

Aptly the method further comprises refining the plurality of possible DNAzyme sequences to modify a length and/or nucleotide composition of a flanking region of each DNAzyme sequence of the plurality of DNAzyme sequence.

Aptly the method further comprises filtering the plurality of possible DNAzyme sequences to remove off-targets.

Aptly the target string comprises an RNA sequence.

Aptly the potential target site comprises a Purine-Pyrimidine junction.

Aptly the predetermined function includes cleaving the target string at the target site.

Aptly the predetermined function probability is a cleaving probability.

Aptly the model is a multiple logistic regression model.

Aptly determining if the predetermined function probability of each DNAzyme sequence of the plurality of DNAzyme sequences of the further group are above a predetermined probability threshold includes performing logistic regression analysis utilising the at least one DNAzyme characteristic of each DNAzyme sequence of the plurality of possible DNAzyme sequences of the further group.

Aptly the at least one DNAzyme characteristic of each DNAzyme sequence of the plurality of possible DNAzyme sequences includes one or more of a pairing free energy between each DNAzyme sequence of the plurality of possible DNAzyme sequences and the target string, an internal structure energy of each DNAzyme sequence of the plurality of possible DNAzyme sequences, a dimer energy between two DNAzyme molecules for each DNAzyme sequence of the plurality of possible DNAzyme sequences and a nucleotide composition of each DNAzyme sequence of the plurality of possible DNAzyme sequences.

Aptly the plurality of possible DNAzyme sequences are ‘10-23’ DNAzymes and optionally comprise a catalytic core of about around 15 nucleotides.

Aptly the method further comprises determining a further DNAzyme characteristic of each DNAzyme sequence of the plurality of possible DNAzyme sequences;

Aptly the further DNAzyme characteristic of each DNAzyme sequence of the plurality of possible DNAzyme sequences includes one of a pairing free energy between each DNAzyme sequence of the plurality of possible DNAzyme sequences and the target string, an internal structure energy of each DNAzyme sequence of the plurality of possible DNAzyme sequences, a dimer energy between two DNAzyme molecules for each DNAzyme sequence of the plurality of possible DNAzyme sequences and a nucleotide composition of each DNAzyme sequence of the plurality of possible DNAzyme sequences.

Aptly the model includes a combination of the at least one DNAzyme characteristic and the further DNAzyme characteristic for each DNAzyme sequence of the plurality of possible DNAzyme sequences, the combination optionally including a weighted sum of the at least one DNAzyme characteristic and the further DNAzyme characteristic for each DNAzyme sequence of the plurality of possible DNAzyme sequences.

Aptly the model includes a weighted sum of the at least one DNAzyme characteristic and the further DNAzyme characteristic for each DNAzyme sequence of the plurality of possible DNAzyme sequences.

Aptly the method is implemented as a computer program stored on non-transitory computer readable storage medium executable on at least one processor-based device.

Aptly the method further comprises training the model using the at least one DNAzyme characteristic of each DNAzyme sequence of the plurality of possible DNAzyme sequences.

Aptly the method further comprises training the model using a still further DNAzyme characteristic of each DNAzyme sequence of the plurality of possible DNAzyme sequences, the further DNAzyme characteristic optionally including a pairing free energy between each DNAzyme sequence of the plurality of possible DNAzyme sequences and the target string, an internal structure energy of each DNAzyme sequence of the plurality of possible DNAzyme sequences, a dimer energy between two DNAzyme molecules for each DNAzyme sequence of the plurality of possible DNAzyme sequences and a nucleotide composition of each DNAzyme sequence of the plurality of possible DNAzyme sequences.

Aptly the method further comprises identifying, from the model, a parameter of each DNAzyme sequence of the plurality of possible DNAzyme sequences which substantially impacts the predetermined function probability of each DNAzyme sequence of the plurality of possible DNAzyme sequences of the first group and/or the DNAzyme sequences of the plurality of possible DNAzyme sequences of the further group.

Aptly the model is trained using a machine learning algorithm.

According a second aspect of the present invention there is provided a method for training computer implemented instructions executable on a processor for providing at least one DNAzyme for performing a predetermined function on a target string, comprising the steps of: identifying at least one potential target site of a target string; proposing a plurality of possible DNAzyme sequences which may perform a predetermined function on at least one target site, a predetermined function probability for each DNAzyme sequence of the plurality of DNAzyme sequences being known; determining at least one DNAzyme characteristic of each DNAzyme sequence of the plurality of possible DNAzyme sequences; and providing a model based on the relationship between the at least one DNAzyme characteristic of each DNAzyme sequence of the plurality of possible DNAzyme sequences and the predetermined function probability of each DNAzyme sequence of the plurality of DNAzyme sequences.

Aptly the method further comprises determining at least one DNAzyme characteristic for a plurality of further DNAzyme sequences, a predetermined function probability of each DNAzyme sequence of the plurality of further DNAzyme sequences not being known; and predicting, using the model, the predetermined function probability of each DNAzyme sequence of the plurality of further DNAzyme sequences.

Aptly the method further comprises obtaining a measured predetermined function probability for each DNAzyme sequence of the plurality of further DNAzyme sequences; and refining the model based on the measured predetermined function probability for each DNAzyme sequence of the plurality of further DNAzyme sequences.

Aptly the model includes a combination of the at least one DNAzyme characteristic and a further DNAzyme characteristic for each DNAzyme sequence of the plurality of possible DNAzyme sequences and optionally the model is a multiple logistic regression model, the combination optionally including a weighted sum of the at least one DNAzyme characteristic and the further DNAzyme characteristic for each DNAzyme sequence of the plurality of possible DNAzyme sequences.

Aptly the model includes a weighted sum of the at least one DNAzyme characteristic and a further DNAzyme characteristic for each DNAzyme sequence of the plurality of possible DNAzyme sequences and optionally the model is a multiple logistic regression model.

Certain embodiments of the present invention provide an efficient method for providing at least one DNAzyme for performing a predetermined function on a target string.

Certain embodiments of the present invention reduce the costs and timescales associated with providing at least one DNAzyme for performing a predetermined function on a target string.

Certain embodiments of the present invention provide a machine learning approach to providing at least one DNAzyme for performing a predetermined function on a target string.

Certain embodiments of the present invention provide a method for rapidly assessing a predetermined function probability associated with each DNAzyme sequence of a large group of DNAzyme sequences.

Certain embodiments of the present invention provide a method for predicting an efficiency of a DNAzyme sequence without a requirement for laboratory testing.

Certain embodiments of the present invention provide a method for automatic prediction of an efficiency of a DNAzyme sequence.

Embodiments of the present invention will now be described hereinafter, by way of example only, with reference to the accompanying drawings in which:

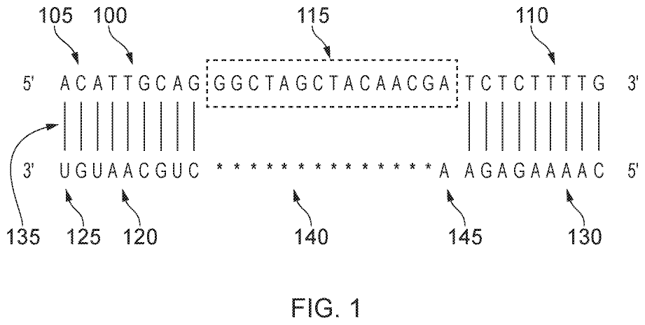

FIG. 1 illustrates an exemplary DNAzyme sequence alongside an exemplary target string;

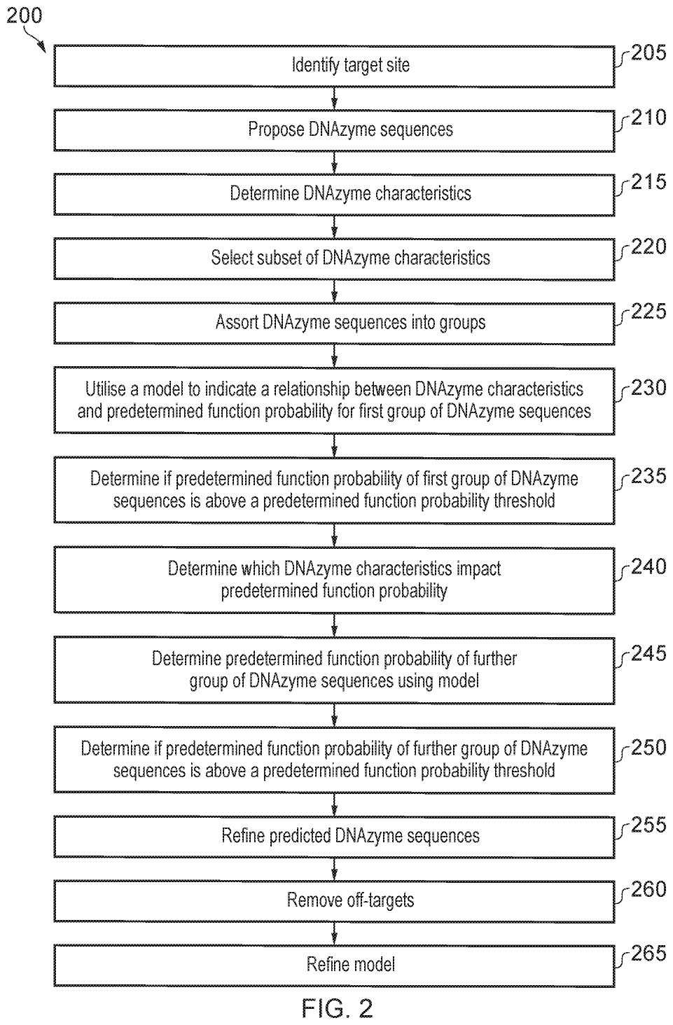

FIG. 2 illustrates a flow diagram of a method for providing at least one DNAzyme for performing a predetermined function on a target string;

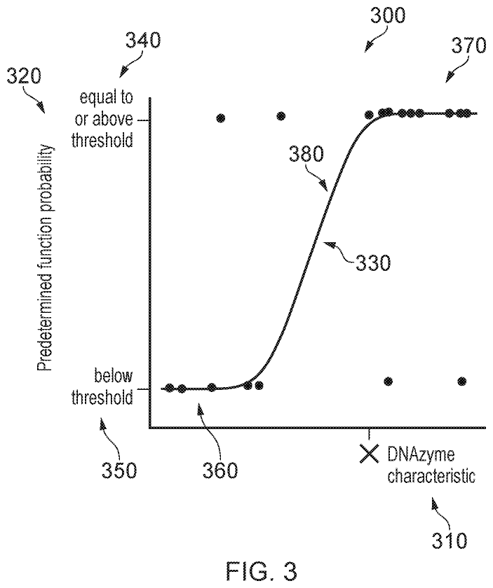

FIG. 3 illustrates an exemplary logistic regression model usable for providing at least one DNAzyme for performing a predetermined function on a target string;

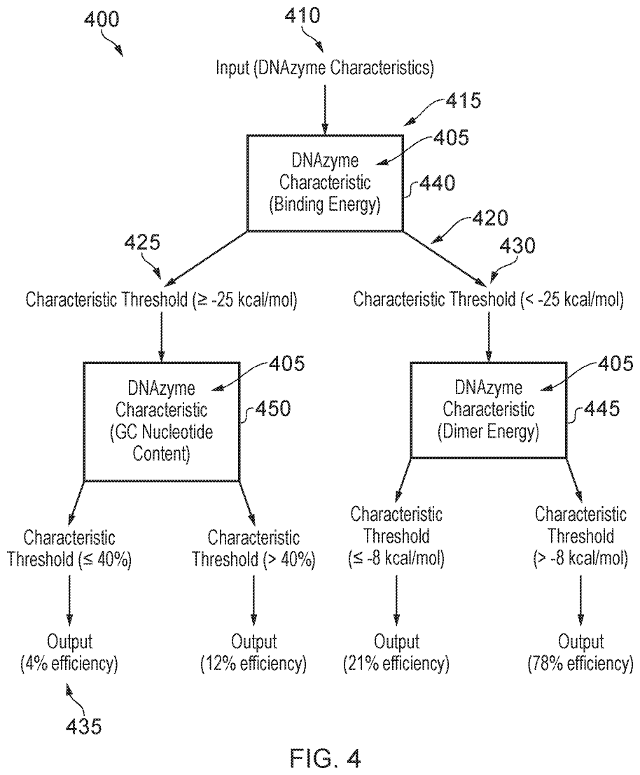

FIG. 4 illustrates an exemplary decision tree model usable for providing at least one DNAzyme for performing a predetermined function on a target string; and

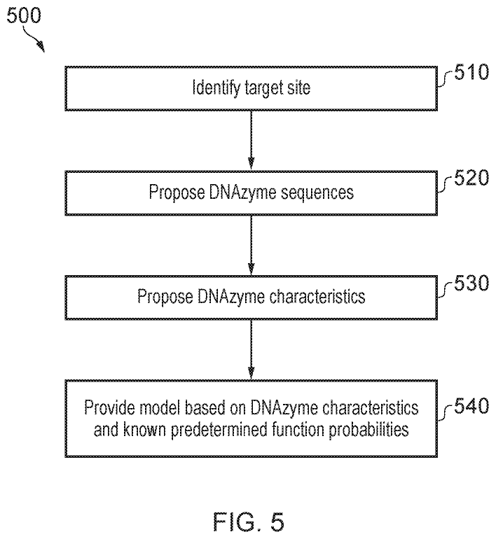

FIG. 5 illustrates a flow diagram for training a method for training a model for providing at least one DNAzyme for performing a predetermined function on a target string.

In the drawings like reference numerals refer to like parts.

FIG. 1 illustrates an exemplary DNAzyme molecule 100. The DNAzyme molecule 100 illustrated in FIG. 1 is a so-called 10-23 DNAzyme. It will be understood that FIG. 1 illustrates a schematic representation of a 10-23 DNAzyme. The DNAzyme 100 of FIG. 1 is a linear chain of nucleotides and includes two arm or flanking regions 105, 110 alongside a catalytic core 115. The catalytic core 115 of the DNAzyme 100 is enclosed by the dotted box in FIG. 1. The flanking regions 105, 110 are arranged on either side of the catalytic core 115 such that the catalytic core 115 is situated between the arm/flanking regions 105, 110 within the linear DNAzyme sequence 100. It will be appreciated that other types of DNAzyme, such as 8-17 DNAzymes, may be envisaged.

The DNAzyme catalytic core 115 is composed of a predetermined linear arrangement of nucleotides. For a 10-23 DNAzyme, this is a nucleotide sequence of 15 nucleotides arranged as 5′-GGCTAGCTACAACGA-3′ and is indicated within the dotted box in FIG. 1. The flanking regions 105, 110 can be of variable length (or contain a variable number of nucleotides) and can include a wide variety of nucleotide arrangements. Each of the two flanking regions 105, 110 may optionally include the same number of nucleotides. Each of the two flanking regions 105, 110 may optionally include an identical sequence of nucleotides. Each of the two flanking regions 105, 110 may optionally include a different sequence of nucleotides. Each of the flanking regions 105, 110 illustrated in FIG. 1 include nine nucleotides, but contain different nucleotide sequences.

FIG. 1 also illustrates an RNA string 120 which can be targeted by the DNAzyme 100 of FIG. 1. The RNA string 120 of FIG. 1 is therefore an example of a target string. That is to say that the DNAzyme 100 nucleotide sequence is arranged such that it complements the nucleotide sequence/arrangement of the RNA string 120. Optionally the DNAzyme nucleotide sequence is specific to target a single RNA string. Optionally the DNAzyme nucleotide sequence is such that a plurality of RNA strings are targetable. The target RNA string is a linear arrangement of nucleotides 120 and, similarly to the DNAzyme molecule 100, includes two flanking/arm regions 125, 130. The flanking regions 125, 135 of the RNA string of FIG. 1 each include 9 nucleotides but contain different nucleotide sequences.

As is illustrated in FIG. 1, the nucleotide sequence of the flanking/arm regions 105, 110 of the DNAzyme molecule 100 are arranged to be complimentary with the flanking/arm regions 125, 130 of the target RNA string 120. That is to say a respective arm region 105 of the DNAzyme 100 has a nucleotide sequence complimentary to that of a respective arm region 125 of the target RNA string 120 and the remaining arm region 110 of the DNAzyme 100 has a nucleotide sequence complimentary to that of the remaining arm region 130 of the target RNA string 120. Each nucleotide in the arm/flanking regions 105, 110 of the DNAzyme 100 thus forms a bond 135 with a complimentary nucleotide at a corresponding position in the arm/flanking regions 125, 130 of the RNA string 120. It will be understood that DNAzymes bind to respective targets via Watson-Crick base pairing. The bonds 135 between complimentary nucleotides in the arm regions 105, 110, 125, 130 of the DNAzyme and the RNA string respectively are indicated in FIG. 1 by the vertical lines.

The target site/cleaving point 140 in the RNA string 120 is indicated with asterisks in FIG. 1. The target site/cleaving point 140 of the RNA string 120 is cleft due to the catalytic core 115 of the DNAzyme. The cleaving point/target site 140 includes a Purine-Pyrimidine junction. Possible Purine-Pyrimidine junctions include 5′-AC-3′, 5′-AU-3′, 5′-GC-3′, 5′-GU-3′ nucleotide arrangements. The Purine 145 of the Purine-Pyrimidine junction of the target site/cleaving point (A in the RNA string of FIG. 1) is unpaired. That is to say that the unpaired purine is not linked, by hydrogen bonding, to a pyrimidine in intramolecular or intermolecular base pairing. The Purine 145 of the Purine-Pyrimidine junction of the target site/cleaving point does not form a bond with the DNAzyme molecule. The unpaired purine at the Purine-Pyrimidine junction creates a “wobble pair”. It will be understood that such a “wobble pair” arrangement promotes cleavage of the target RNA via a de-esterification process in which phosphodiester bonds linking the RNA bases are broken. The RNA molecule is thus broken into two (or more) parts at the site of the Purine-Pyrimidine junction.

FIG. 2 illustrates a method 200 for providing at least one DNAzyme for performing a predetermined function on a target string. Optionally the target string is an RNA string. Optionally the RNA string includes a Purine-Pyrimidine junction. Optionally the predetermined function is cleaving the target string at a target location/site. It will be understood that prior to the method illustrated in FIG. 2, and further described below, the target string for which a compatible/complimentary DNAzyme is desired is determined/identified.

At a first step s205 of the method 200, at least one potential target site of the target string is identified. It will be understood that the target site is a region of the target string upon which a DNAzyme can act to provide a desired effect/function. It will also be understood that a target string may include a single target site. Alternatively, a target string may include a plurality of target sites. Optionally only a single target site, or a group/selection of possible target sites are identified. Optionally all possible target sites of the target string are identified. Optionally the target site includes a Purine-Pyrimidine junction. It will be understood that a single target string, for an example an RNA string, may include many (tens or hundreds or more) Purine-Pyrimidine junctions. As indicated above Purine-Pyrimidine junctions include 5′-AC-3′, 5′-AU-3′, 5′-GC-3′, 5′-GU-3′ nucleotide arrangements in a target string. Optionally all Purine-Pyrimidine junctions in a target site are identified.

At a next step s210 of the method 200, a plurality of possible DNAzyme sequences which may perform a predetermined function on the target site are proposed. It will be understood that the proposed DNAzyme sequences are complimentary in respect of at least a portion of the target string and can therefore interact with at least a portion of the target string. For example, regions (such as arm/flanking regions) of each DNAzyme may be arranged to bind to particular regions (such as arm/flanking regions) of the target string. It will be understood that a plurality of DNAzyme sequences may be proposed for each identified target site of the target string. Optionally tens or hundreds or thousands or more potential/possible DNAzyme sequences are proposed for each target site of the target string.

The proposed DNAzyme sequences each include three components, as is described in relation to FIG. 1 above. The proposed DNAzyme sequences each include a first arm of length n nucleotides that has pairwise complementarity with the n nucleotides in the target string sequence. If the target string is an RNA string including a target site that is a Purine-Pyrimidine junction, it will be understood that the first arm extends from the pyrimidine in the Purine-Pyrimidine junction to the next n−1 contiguous nucleotides towards the 3′ end of the RNA molecule. The proposed DNAzyme sequences also include a catalytic core which provides the enzymatic/cleaving capability of the DNAzyme. If the DNAzyme sequence is a 10-23 DNAzyme, the catalytic core includes the nucleotide sequence of 5′-GGCTAGCTACAACGA-3′. The proposed DNAzyme sequences each additionally include a further arm of length n nucleotides that has pairwise complementarity with the n nucleotides in the target string sequence. If the target string is an RNA string including a target site that is a Purine-Pyrimidine junction, it will be understood that the further arm extends from the nucleotide neighbouring the purine in the Purine-Pyrimidine junction to the next n−1 contiguous nucleotides towards the 5′ end of the RNA molecule. Optionally the length of the first and further arms is nine nucleotides (n=9). Optionally the length of the first and further arms is between one and ten nucleotides. Optionally the length of the first and further arms is any other number of nucleotides.

At a next step s215 of the method 200, at least one DNAzyme characteristic of each of the proposed DNAzyme sequences is determined. Optionally the characteristic may be determined through laboratory experiments. Optionally the characteristic may be determined by utilisation of a mathematical model. Optionally the characteristic may be taken from literature. The DNAzyme characteristic may optionally include a pairing free energy/binding energy between each DNAzyme sequence and the target string. A pairing free energy may optionally be determined using the nearest neighbour method. The DNAzyme characteristic may optionally include potential internal structure energies of each DNAzyme sequence. The potential internal structure energies may optionally be determined using RNAfold computer program/web server and by selecting the DNA option within the program/server. The DNAzyme characteristic may optionally include potential dimer energies between DNAzyme molecules. It will be understood that homodimers can form between pairs of identical DNAzymes where their complementary sequences have partial complementarity with each other. Such dimerization therefore reduces the availability of free DNAzyme molecules and hence an efficiency of the DNAzyme sequence, for example a cleaving efficiency. Potential dimer energies may optionally be determined using RNAup computer program/web server and by selecting the DNA sequence option within the program/server. The at least one DNAzyme characteristic may optionally include information relating to the nucleotide composition of each DNAzyme sequence. Optionally the nucleotide composition information may include the combined proportion of C nucleotides and G nucleotides in the DNAzyme sequence relative to the number of nucleotides of the DNAzyme sequence. Optionally the nucleotide composition information may include the single nucleotide proportions, for example the proportion of each one of the four nucleotides A, C, G and T in the potential/predicted DNAzyme sequence relative to the number of nucleotides in the DNAzyme sequence.

At optional step s220 of the method 200, a subset of the proposed DNAzyme sequences are selected for which a predetermined function probability for each selected DNAzyme sequence will be evaluated/utilised as detailed below. Optionally the evaluation includes determining/predicting a predetermined function probability associated with each selected DNAzyme sequence based on the determined at least one DNAzyme characteristic.

Optionally the evaluation includes extracting a relationship between the determined at least one DNAzyme characteristic and a predetermined function probability for each DNAzyme sequence. Optionally the predetermined function probability is an efficiency of the proposed DNAzyme sequences. Optionally the efficiency represents a proportion of a population of the target string that a given quantity of the DNAzyme is expected to cleave. In vitro cleavage reactions may be performed to assess DNAzyme efficiency. Cleavage reactions may optionally be performed using DNAzymes and RNA substrates at a ratio of 10:1 (10 μM DNAzyme to 1 μM RNA). Reactions may optionally be performed at 37° C. for 60 min in reaction buffer (50 mM Tris-HCl, pH 7,5, 150 mM NaCl, 10 mM MgCl2, 0.01% SDS), and cleavege may be quantified using UREA-Page and densitometry. The subset may optionally be determined manually by a user. The subset may optionally be determined automatically. The subset may optionally be based on structural features of the proposed DNAzyme sequences.

At the next step s225 of the method 200, the proposed DNAzyme sequences are assorted into a plurality of groups of DNAzyme sequences including a first group of DNAzyme sequences and a further group of DNAzyme sequences. The first group of DNAzyme sequences includes the DNAzyme sequences for which a predetermined function probability of each DNAzyme sequence is known. The predetermined function probability of each DNAzyme sequence of the first group of DNAzyme sequences may optionally be retrieved from literature or may optionally be obtained by measurement, for example in a laboratory. The further group of DNAzyme sequences includes the DNAzyme sequences for which a predetermined function probability is unknown. Optionally the predetermined function probability is an efficiency of each DNAzyme sequence. Optionally the efficiency represents a proportion of a target string population that a given quantity of a proposed DNAzyme is expected to cleave.

At a next step s230 of the method 200 a model is utilised to indicate a relationship between the at least one DNAzyme characteristic (determined at step s215 of the method 200) and the known predetermined function probability for each proposed DNAzyme sequence of the first group of DNAzyme sequences. The model is optionally a logistic regression model (if a single DNAzyme characteristic for each DNAzyme sequence of the first group is utilised) or a multiple logistic regression model (if a plurality of DNAzyme characteristics are utilised for each DNAzyme sequence of the first group are utilised) and is fitted to the at least one DNAzyme characteristic of each proposed DNAzyme sequences of the first group alongside the known predetermined function probability of each proposed DNAzyme of the first group. In this sense the first group of proposed DNAzyme sequences can be considered as training data to train the model. It will be appreciated that a subsequent group of proposed DNAzyme sequences for which a predetermined function probability is known can be applied to the model alongside at least one DNAzyme characteristic determined for each of the DNAzyme sequences of this subsequent group to refine the model.

Alternatively, the model may be a machine learning model. Optionally the machine learning model is a decision tree model. Optionally the decision tree model is a regression tree model. The machine learning model is provided with the at least once DNAzyme characteristic (determined at step s215 of the method 200) of each proposed DNAzyme the first group of DNAzyme sequences alongside the known predetermined function probability of each proposed DNAzyme sequence of the first group. The first group of proposed DNAzyme sequences is thus training data allowing the machine learning model to infer a dependence of a predetermined function probability on at least one DNAzyme characteristic for a given DNAzyme sequence. As the machine learning model is iterated in use, the accuracy/reliability of the model is improved.

It will be understood that, instead of a single model, both a logistic regression/multiple logistic regression model and a machine learning model may optionally be utilised. Optionally two, three, or more models are utilised. In such a case, both the logistic regression/multiple logistic regression model and the machine learning model are each independently trained using the proposed DNAzyme sequences of the first group as is described in the above paragraphs. A benefit of utilising two (or more) different models, including a logistic regression/multiple logistic regression model and a machine learning model, is that predetermined function probabilities returned by each model, the predetermined function probabilities being predicted/determined based on a characteristic (or characteristics) of respective DNAzyme sequences, can be compared as an indicator of reliability. The logistic regression/multiple logistic regression model approach provides a robust model whereas some machine learning models, such as decision tree models, may initially be prone to overfitting. Therefore, a comparison between two or more models may help increase model reliability. It will also be appreciated that use of a multiple logistic regression model (utilising a plurality of determined DNAzyme characteristics for each proposed DNAzyme sequence) can help indicate the importance/impact of each DNAzyme characteristic on the predetermined function probability. A decision tree model can identify potentially complex relationships between a plurality of DNAzyme characteristics. The use of these two models in tandem therefore allows for greater predictive power in designing suitable DNAzyme sequences for a particular target string.

At a next step s235 of the method 200, using the model which optionally is a logistic regression model/multiple logistic regression model and/or a machine learning model, it is determined if the predetermined function probability of each DNAzyme of the first group is above a predetermined function probability threshold. That is to say that the model optionally returns one of two possible outcomes for each proposed DNAzyme sequence of the first group, a result of zero (0) when the predetermined function probability of a respective DNAzyme sequence of the first group is below the predetermined function probability threshold and a result of one (1) when then predetermined function probability of a respective DNAzyme sequence of the first group is above or equal to the predetermined function probability threshold. The outcome/output of the model can therefore be considered to be binary/digital. The predetermined function probability threshold is optionally defined by a user. The predetermined function probability threshold is optionally an efficiency threshold. The efficiency threshold optionally represents a threshold proportion of target string population that a given quantity of a proposed DNAzyme sequence is expected to cleave. Optionally the threshold efficiency is 40%. Optionally the threshold efficiency is 50%.

As an optional step s240 of the method, if multiple DNAzyme characteristics were determined for each DNAzyme sequence at step 3 215 of the model 200, it is identified which of the at least one DNAzyme characteristics of each DNAzyme of the first group, impact or substantially impact the predetermined function probability of the DNAzymes. This is optionally achieved by producing an ANOVA table. An example of an ANOVA table is shown in Table 1 below. Identifying DNAzyme characteristics that have the largest impact on the predetermined function probability may aid in the proposal of future DNAzyme sequences and in the selection of suitable DNAzyme sequences to target a particular target string.

TABLE 1

| | | | | Residual | |
|---|---|---|---|---|---|
| | df | Deviance | Residual df | Deviance | p-value |
| Null | | | 37 | 45.7 | |
| Binding Energy | 1 | 6.3 | 36 | 39.4 | 0.011 |
| Dimer Energy | 1 | 0.8 | 35 | 38.5 | 0.825 |
| Internal Energy | 1 | 4.2 | 34 | 34.3 | 0.040 |

At another step s245 of the method 200, a predetermined function probability is determined for each of the DNAzyme sequences of the second group of DNAzyme sequences by applying the at least one characteristic determined for each DNAzyme (determined at step s215 of the model 200) of the second group to the model. It will be understood that the model was previously fit to the at least one characteristic of each DNAzyme sequence of the first group alongside the known predetermined function probability of each DNAzyme of the first group to indicate a relationship between the at least one DNAzyme characteristic and the predetermined function probability of a given DNAzyme sequence. The model can thus determine/predict a predetermined function probability for each DNAzyme of the second group based on the at least one characteristic of each DNAzyme sequence of the second group. The predetermined function probability optionally is a predicted efficiency and can be referred to as a probability of success of a DNAzyme. The probability of success optionally represents a proportion of target string population that a given quantity of a proposed DNAzyme is expected to cleave.

At another step s250 of the method 200, using the model which optionally is a logistic regression model/multiple logistic regression model and/or a machine learning model, it is determined if the predetermined function probability of each DNAzyme of the second group is above a predetermined function probability threshold. That is to say that the model optionally returns one of two possible outcomes for each proposed DNAzyme sequence of the second group, a result of zero (0) when the predetermined function probability of a respective DNAzyme sequence of the second group is below the predetermined function probability threshold and a result of one (1) when then predetermined function probability of a respective DNAzyme sequence of the second group is above or equal to the predetermined function probability threshold. The outcome/output of the model can therefore be considered to be binary/digital. The predetermined function probability threshold is optionally defined by a user. The predetermined function probability threshold is optionally an efficiency threshold. The efficiency threshold optionally represents a threshold proportion of target string population that a given quantity of a proposed DNAzyme sequence is expected to cleave. Optionally the threshold efficiency is 40%. Optionally the threshold efficiency is 50%.

It will be appreciated that if multiple models including a logistic regression/multiple logistic regression model and a machine leaning model are utilised, the logistic regression model can report, with relatively robust reliability, if a plurality of proposed DNAzyme sequences are above a predetermined function probability threshold or not. The machine learning model can rank the DNAzyme sequences deemed to be above the predetermined function probability threshold based on the reported predetermined probability function for each sequence. Thus, the use of a logistic regression/multiple logistic regression model and a machine leaning model in tandem allows for the ranking of proposed DNAzyme sequences based on their suitability in respect of a particular target string while offering a degree of reliability that the ranked DNAzyme sequences are indeed appropriate for use in respect of the particular target string. Furthermore, should one model determine a particular DNAzyme sequence to be suitable and a further model determine the same DNAzyme sequence to be unsuitable in respect of the target string, this can be flagged to a user. The use of two models in tandem therefore allows for filtration of DNAzyme sequences mistakenly deemed to be suitable by a particular model.

Via an optional step s255 of the method 200, the predicted DNAzymes can be refined. This optionally includes modifying the length of the arm/flanking regions of the proposed DNAzyme sequences (by modifying number of nucleotides within the arm/flanking regions). It will be appreciated that longer flanking/arm regions may increase the specificity of a DNAzyme to a particular target string and also may increase the binding energy between the DNAzyme are the target string. Longer flanking/arm regions may however result in the development of internal structures within the DNAzyme which may interfere with desired interactions between the DNAzyme and the target string. Optionally the length of each arm/flanking region is set to nine nucleotides. Optionally the length of the arm/flanking region is incrementally reduced by one nucleotide until the internal structure energy of the DNAzyme molecule does not increase significantly (optionally by 1 kcal/mol). Optionally the length of the arm/flanking region is incrementally reduced by one nucleotide until the pairing energy between the DNAzyme molecule and the target string decreases significantly (optionally by 1 kcal/mol).

At another optional step s260 of the method 200, off-targets are identified and removed. Off targets in the context of the present application are proposed DNAzyme sequences that can potentially target/are compatible with strings that are not the target string. That is to say that off targets are not specific to the target string. Optionally off targets can be identified using a set of transcripts which are optionally from a database. The database may optionally be the GENCODE database. The database may optionally be the ENSEMBL database. It would be understood that any other suitable database may be used. Optionally off targets can be identified from custom sequences.

At another optional step s265 of the method 200, the model is refined. This can optionally be achieved by testing DNAzymes determined as suitable by the model using full-length RNA degradation assays and incorporating the results into the model. Optionally a new model can be provided which utilises all of the DNAzyme sequences of the first and second groups as training data for the new model.

FIG. 3 illustrates a logistic regression model 300 usable for indicating a relationship between a DNAzyme characteristic 310 and a predetermined function probability 320. The DNAzyme characteristic may optionally include a pairing free energy/binding energy between each DNAzyme sequence and the target string. The DNAzyme characteristic may optionally include potential internal structure energies of each DNAzyme sequence. The DNAzyme characteristic may optionally include potential dimer energies between DNAzyme molecules. The DNAzyme characteristic may optionally include information relating to the nucleotide composition of each DNAzyme sequence. Optionally the nucleotide composition information may include the combined proportion of C nucleotides and G nucleotides in the DNAzyme sequence relative to the number of nucleotides of the DNAzyme sequence. Optionally the nucleotide composition information may include the single nucleotide proportions, for example the proportion of each one of the four nucleotides A, C, G and T in the potential/predicted DNAzyme sequence relative to the number of nucleotides in the DNAzyme sequence. Optionally the predetermined function 310 probability is an efficiency of a respective DNAzyme sequence, the efficiency representing a proportion of a target string population that a given quantity of the DNAzyme sequence is expected to cleave. The logistic regression model 300 of FIG. 3 is initially trained using training data relating to a plurality of training DNAzyme sequences. The training data includes at least one DNAzyme characteristic determined for each DNAzyme sequence of the training data and a known predetermined function probability for each DNAzyme sequence of the training data. Following training of the logistic regression model 300, the model 300 can be used to predict/determine the predetermined function probability of DNAzyme sequences based on the characteristic determined for each of the DNAzyme sequences.

The logistic regression of FIG. 3 is a statistical model that fits a logistic/sigmoid function 330 to the DNAzyme characteristic for each DNAzyme sequence order to provide a binary/digital output for each DNAzyme sequence. The output for each DNAzyme sequence is either that the predetermined function probability of a respective DNAzyme sequence is greater than or equal to a predetermined function probability threshold 340 or that a predetermined function probability of a respective DNAzyme sequence is less than a predetermined function probability threshold 350. In essence, the logistic/sigmoid function 330 provides a first region 360 including values of the DNAzyme characteristic that do not yield a predetermined function probability greater than or equal to a predetermined function probability threshold, a further region 370 including values of the DNAzyme characteristic that yield a predetermined function probability that is greater than or equal to a predetermined probability threshold and a transition region 380. For example, characteristic X indicated in FIG. 3 would be deemed to yield a predetermined function probability to be greater than or equal to a predetermined function probability threshold.

It will be appreciated that a similar principle applies for a multiple logistic regression model wherein a plurality of DNAzyme characteristics are assessed for each DNAzyme sequence. In essence, the multiple logistic regression model determines if the combination of the plurality of DNAzyme characteristics of each proposed DNAzyme sequence yields a predetermined function probability that is greater than or equal to a predetermined function probability threshold, or not. Optionally the combination of the plurality of DNAzyme characteristics is the weighted sum of the plurality of DNAzyme characteristics. An exemplary multiple logistic regression model, using exemplary DNAzyme characteristics is provided by:

$$log ( p / ( 1 - p ) ) = β 0 + β1 * [ RNA / DNA energy ] + β2 * [ Internal structure ] + β3 * [ Dimer energy ] + β4 * [ CG content ] + β5 * [ C content ] + β6 * [ A content ] + interaction\_terms + error\_term .$$

Here, p is the predetermined function probability. It will be appreciated that any suitable DNAzyme characteristics may be incorporated. In this example the binding energy between the proposed DNAzyme sequence and a target RNA string, the internal structure of the DNAzyme molecule, a dimer energy between two DNAzyme molecules, the CG nucleotide content/composition, C nucleotide content/composition and A nucleotide content/composition are included as DNAzyme characteristics. It will thus be appreciated that a multiple logistic regression model can reveal which of these DNAzyme characteristics substantially effect the resulting predetermined function probability of a DNAzyme sequence.

FIG. 4 illustrates a decision tree model 400 usable for providing at least one DNAzyme for performing a predetermined function on a target string. It will be understood that the decision tree 400 of FIG. 4 is a regression tree. Utilisation of a decision tree model requires that multiple DNAzyme characteristics are determined for each proposed DNAzyme sequence. After a respective DNAzyme sequence is proposed, and a plurality of DNAzyme characteristics 405 are determined for the DNAzyme, the DNAzyme information is provided to the decision tree model 400 as an input 410. The DNAzyme characteristics may optionally include a pairing free energy/binding energy between each DNAzyme sequence and the target string. The DNAzyme characteristics may optionally include potential internal structure energies of each DNAzyme sequence. The DNAzyme characteristics may optionally include potential dimer energies between DNAzyme molecules. The DNAzyme characteristics may optionally include information relating to the nucleotide composition of each DNAzyme sequence. Optionally the nucleotide composition information may include the combined proportion of C nucleotides and G nucleotides in the DNAzyme sequence relative to the number of nucleotides of the DNAzyme sequence. Optionally the nucleotide composition information may include the single nucleotide proportions, for example the proportion of each one of the four nucleotides A, C, G and T in the potential/predicted DNAzyme sequence relative to the number of nucleotides in the DNAzyme sequence.

As is illustrated in FIG. 4, each of the determined DNAzyme characteristics form nodes 415 of the decision tree 400. The decision tree partitions each DNAzyme characteristic into dichotomies 420 whereby a characteristic threshold value of each characteristic provides branches 425, 430. For each node 415 of the tree 400 of FIG. 4 there is therefore two resulting branches 425, 430, a first branch 425 whereby the characteristic is equal to or greater than the characteristic threshold and a further branch 430 whereby the characteristic is less than the characteristic threshold. The characteristic threshold therefore determines the subsequent node of the tree for a particular DNAzyme sequence and therefore determines which of the DNAzyme characteristics are to be evaluated. Following evaluation of a particular path of nodes of the decision tree model, a predetermined function probability is returned by the decision tree model as an output 435. Optionally, the decision tree model is subsequently iterated using the same DNAzyme sequence but evaluating the DNAzyme characteristics 405 in a different order. Optionally the decision tree model is subsequently iterated using a different DNAzyme sequence. Optionally the characteristic threshold is provided by a user. Optionally the characteristic threshold is provided by the decision tree model. Optionally the characteristic threshold is refined as the decision tree model is iterated such that the decision tree model becomes more accurate with use. The decision tree model can therefore be considered a machine learning model.

In the regression tree 400 of FIG. 4, a first node 440 receives the input data 405. The first node computes if a binding energy of a proposed DNAzyme sequence with a target string is equal to/greater than or less than a characteristic threshold of −25 kcal/mol in FIG. 4. The regression tree 400 of FIG. 4 also includes a further node 445 that computes if a dimer energy of a proposed DNAzyme is greater than to or less than/equal to a characteristic threshold of −8 kcal/mol. It will be appreciated that the regression tree 400 of FIG. 4 progresses to the further node if the binding energy is determined to be below −25 kcal/mol at the first node. The regression tree 400 of FIG. 4 also includes a still further node 450 that computes if a GC nucleotide content of a proposed DNAzyme sequence is greater than to or less than/equal to a characteristic threshold of 40%. It will be understood that the regression tree 400 of FIG. 4 progresses to the still further node 450 if the binding energy is determined to be greater than/equal to −25 kcal/mol at the first node 440. Four predetermined function probability outputs 435, that predicted efficiencies of the proposed DNAzyme sequence, are indicated and are responsive to the path taken throughout the decision tree. It will be understood that all DNAzyme characteristics, characteristic thresholds and outputs illustrated in the regression tree 400 of Figure for are exemplary only. Another suitable threshold level and/or rationale for assessing if a threshold is met can of course be utilised rather than those explicitly mentioned. It will be understood that any suitable DNAzyme characteristic can be utilised in any node of a decision tree, that any suitable characteristic threshold can be utilised in any branch of the decision tree and that the output from the decision tree depends on, and varies with, these factors/variables.

FIG. 5 illustrates a method for training a model for providing at least one DNAzyme for performing a predetermined function on a target string. Optionally the method is for training computer implemented instructions executable on a processor for providing at least one DNAzyme for performing a predetermined function on a target string.

At a first step s510 of the method 500 of FIG. 5 at least one target site on a target string is identified. As discussed, a target string is optionally an RNA string and a target site is optionally a Purine-Pyrimidine junction.

At a second step s520 of the method, for each target site, a plurality of DNAzyme sequences are proposed which may perform a predetermined function on/at the target site of the target string. Optionally the DNAzyme includes a catalytic core which is able to cleave the target string at the target site, for example at a Purine-Pyrimidine junction. The DNAzyme sequences are such that at least a portion of the DNAzyme is complimentary, and interacts with/binds to, at least a portion of the target string. Optionally two, or more, arm regions of the DNAzyme sequence interact with two, or more, respective arm regions of the target string.

At a third step s530 of the method 500 at least one DNAzyme characteristic is determined for each of the proposed DNAzyme sequences. The DNAzyme characteristic may optionally include a pairing free energy/binding energy between each DNAzyme sequence and the target string. The DNAzyme characteristic may optionally include potential internal structure energies of each DNAzyme sequence. The DNAzyme characteristic may optionally include potential dimer energies between DNAzyme. The at least one DNAzyme characteristic may optionally include information relating to the nucleotide composition of each DNAzyme sequence. Optionally the nucleotide composition information may include the combined proportion of C nucleotides and G nucleotides in the DNAzyme sequence relative to the number of nucleotides of the DNAzyme sequence. Optionally the nucleotide composition information may include the single nucleotide proportions, for example the proportion of each one of the four nucleotides A, C, G and T in the potential/predicted DNAzyme sequence relative to the number of nucleotides in the DNAzyme sequence.

At a fourth step s540 of the method 500, a model is utilised and applied to all proposed DNAzymes sequences for which a predetermined function probability is known. The predetermined function probability can optionally be obtained from laboratory experiments. The predetermined function probability can optionally be determined from literature. The predetermined probability function is optionally an efficiency of a DNAzyme sequence whereby the efficiency represents the percentage proportion of a target string population that a particular quantity of the DNAzyme is expected to cleave. The model is applied to the at least one DNAzyme characteristic determined at the third step s530 of the method 500 of FIG. 5 alongside the known predetermined function probability for each of the proposed DNAzyme sequences for which the predetermined function probability is known, respectively. Thus, a relationship between the at least one DNAzyme characteristic determined at the third step s530 of the method 500 illustrated in FIG. 5 and the predetermined function probability of respective DNAzyme sequences can be indicated. Thus, the model is provided based on the relationship between the at least one DNAzyme characteristic and the predetermined function probability. Optionally the model is a logistic regression model. Optionally the model is a multiple logistic regression model. Optionally the model is a decision tree model. Optionally the model is a regression tree model. Optionally the model is a machine learning model.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method for providing at least one DNAzyme for performing a predetermined function on a target string, comprising the steps of:
identifying at least one potential target site of a target string;
proposing a plurality of possible DNAzyme sequences which may perform a predetermined function on at least one target site;
determining at least one DNAzyme characteristic of each DNAzyme sequence of the plurality of possible DNAzyme sequences;
utilising a model to indicate a relationship between the at least one DNAzyme characteristic and a predetermined function probability of each DNAzyme sequence of the plurality of possible DNAzyme sequences; and
determining if the predetermined function probability each DNAzyme sequence of the plurality of possible DNAzyme sequences are above a predetermined function probability threshold.

2. The method as claimed in claim 1, further comprising:
assorting the plurality of possible DNAzyme sequences into a plurality of groups whereby a first group includes DNAzyme sequences of the plurality of DNAzyme sequences for which the predetermined function probability is known, and a further group includes DNAzyme sequences of the plurality of DNAzyme sequences for which the predetermined function probability is unknown.

3. The method as claimed in claim 2, whereby:
determining if the predetermined function probability of each DNAzyme sequence of the plurality of possible DNAzyme sequences of the first group are above a predetermined function probability threshold includes fitting the model to the at least one DNAzyme characteristic of each DNAzyme sequence of the plurality of possible DNA sequences of the first group and the predetermined function probability of the plurality of possible DNAzyme sequences of the first group.

4. The method as claimed in claim 2, wherein:
determining if the predetermined function probability each DNAzyme sequence of the plurality of possible DNAzyme sequences of the further group are above a predetermined function probability threshold includes providing the at least one DNAzyme characteristic of each DNAzyme sequence of the plurality of possible DNAzyme sequences of the first group to the model to determine the predetermined function probability of each DNAzyme sequence of the plurality of possible DNAzyme sequences of the further group.

5. The method as claimed in claim 1, further comprising:
refining the plurality of possible DNAzyme sequences to modify a length and/or nucleotide composition of a flanking region of each DNAzyme sequence of the plurality of DNAzyme sequence.

6. The method as claimed in claim 1, further comprising:
filtering the plurality of possible DNAzyme sequences to remove off-targets.

7. (canceled)

8. The method as claimed in claim 1, wherein:
the potential target site comprises a Purine-Pyrimidine junction, and/or the
target string comprises an RNA sequence.

9. The method as claimed in claim 1, wherein:
the predetermined function includes cleaving the target string at the target site, and/or
the predetermined function probability is a cleaving probability.

10. (canceled)

11. (canceled)

12. The method as claimed in claim 4, wherein:
the model is a multiple logistic regression model, and determining if the predetermined function probability of each DNAzyme sequence of the plurality of DNAzyme sequences of the further group are above a predetermined probability threshold includes performing logistic regression analysis utilising the at least one DNAzyme characteristic of each DNAzyme sequence of the plurality of possible DNAzyme sequences of the further group.

13. The method as claimed in claim 1, wherein:
the at least one DNAzyme characteristic of each DNAzyme sequence of the plurality of possible DNAzyme sequences includes one or more of a pairing free energy between each DNAzyme sequence of the plurality of possible DNAzyme sequences and the target string, an internal structure energy of each DNAzyme sequence of the plurality of possible DNAzyme sequences, a dimer energy between two DNAzyme molecules for each DNAzyme sequence of the plurality of possible DNAzyme sequences and a nucleotide composition of each DNAzyme sequence of the plurality of possible DNAzyme sequences.

14. The method as claimed in claim 1, further comprising:
the plurality of possible DNAzyme sequences are ‘10-23’ DNAzymes and optionally comprise a catalytic core of about around 15 nucleotides.

15. The method as claimed in claim 1, further comprising:
determining a further DNAzyme characteristic of each DNAzyme sequence of the plurality of possible DNAzyme sequences, the further DNAzyme characteristic of each DNAzyme sequence of the plurality of possible DNAzyme sequences optionally including one of a pairing free energy between each DNAzyme sequence of the plurality of possible DNAzyme sequences and the target string, an internal structure energy of each DNAzyme sequence of the plurality of possible DNAzyme sequences, a dimer energy between two DNAzyme molecules for each DNAzyme sequence of the plurality of possible DNAzyme sequences and a nucleotide composition of each DNAzyme sequence of the plurality of possible DNAzyme sequences.

16. (canceled)

17. The method as claimed in claim 12, wherein:
the model includes a combination of the at least one DNAzyme characteristic and the further DNAzyme characteristic for each DNAzyme sequence of the plurality of possible DNAzyme sequences, the combination optionally including a weighted sum of the at least one DNAzyme characteristic and the further DNAzyme characteristic for each DNAzyme sequence of the plurality of possible DNAzyme sequences.

18. The method as claimed in claim 1, wherein:
the method is implemented as a computer program stored on non-transitory computer readable storage medium executable on at least one processor-based device.

19. The method as claimed in claim 14, further comprising
training the model using the at least one DNAzyme characteristic of each DNAzyme sequence of the plurality of possible DNAzyme sequences; whereby
the model is optionally trained using a machine learning algorithm.

20. The method as claimed in claim 15, further comprising:
training the model using a still further DNAzyme characteristic of each DNAzyme sequence of the plurality of possible DNAzyme sequences, the further DNAzyme characteristic optionally including a pairing free energy between each DNAzyme sequence of the plurality of possible DNAzyme sequences and the target string, an internal structure energy of each DNAzyme sequence of the plurality of possible DNAzyme sequences, a dimer energy between two DNAzyme molecules for each DNAzyme sequence of the plurality of possible DNAzyme sequences and a nucleotide composition of each DNAzyme sequence of the plurality of possible DNAzyme sequences; and/or
identifying, from the model, a parameter of each DNAzyme sequence of the plurality of possible DNAzyme sequences which substantially impacts the predetermined function probability of each DNAzyme sequence of the plurality of possible DNAzyme sequences of the first group and/or the DNAzyme sequences of the plurality of possible DNAzyme sequences of the further group.

21. (canceled)

22. (canceled)

23. A method for training computer implemented instructions executable on a processor for providing at least one DNAzyme for performing a predetermined function on a target string, comprising the steps of:
identifying at least one potential target site of a target string;
proposing a plurality of possible DNAzyme sequences which may perform a predetermined function on at least one target site, a predetermined function probability for each DNAzyme sequence of the plurality of DNAzyme sequences being known;
determining at least one DNAzyme characteristic of each DNAzyme sequence of the plurality of possible DNAzyme sequences; and
providing a model based on the relationship between the at least one DNAzyme characteristic of each DNAzyme sequence of the plurality of possible DNAzyme sequences and the predetermined function probability of each DNAzyme sequence of the plurality of DNAzyme sequences.

24. The method as claimed in claim 23, further comprising:
determining at least one DNAzyme characteristic for a plurality of further DNAzyme sequences, a predetermined function probability of each DNAzyme sequence of the plurality of further DNAzyme sequences not being known; and
predicting, using the model, the predetermined function probability of each DNAzyme sequence of the plurality of further DNAzyme sequences.

25. The method as claimed in claim 24, further comprising:
obtaining a measured predetermined function probability for each DNAzyme sequence of the plurality of further DNAzyme sequences; and
refining the model based on the measured predetermined function probability for each DNAzyme sequence of the plurality of further DNAzyme sequences.

26. The method as claimed in claim 23, wherein:
the model includes a combination of the at least one DNAzyme characteristic and a further DNAzyme characteristic for each DNAzyme sequence of the plurality of possible DNAzyme sequences and optionally the model is a multiple logistic regression model, the combination optionally including a weighted sum of the at least one DNAzyme characteristic and the further DNAzyme characteristic for each DNAzyme sequence of the plurality of possible DNAzyme sequences.
